(19) United States
(12) Patent Application Publication
(10) Pub. No.: US 2012/0064528 A1
(43) Pub. Date: Mar. 15, 2012

(54) METHODS AND REAGENTS FOR QUANTIFYING NUCLEIC ACID FRAGMENTATION AND APOPTOSIS (QLM-PCR, CELL NUMBER QPCR AND APOQPCR)

(75) Inventor: DAVID JAMES HOOKER, MELBOURNE (AU)

(73) Assignee: THE MACFARLANE BURNET INSTITUTE FOR MEDICAL RESEARCH AND PUBLIC HEALTH LTD, Melbourne (AU)

(21) Appl. No.: 13/151,098

(22) Filed: Jun. 1, 2011

Related U.S. Application Data
(60) Provisional application No. 61/350,419, filed on Jun. 1, 2010.

(51) Int. Cl. C12Q 1/68; C07H 21/04

(52) U.S. Cl. 435/6.12; 536/23.1

(57) ABSTRACT

A method for quantifying apoptosis in absolute terms in a cellular sample by real-time ligation-mediated PCR, the method comprising: (a) obtaining first control genomic nucleic acid which is apoptotic nucleic acid from a cellular sample that is substantially 100% apoptotic; (b) obtaining test genomic nucleic acid derived from a test cellular sample; (c) subjecting a plurality of different known concentrations of first control genomic nucleic acid to real-time apoptosis-specific multi-product PCR in the presence of a nucleic acid-binding fluorophore or other detectable tag to obtain a threshold cycle number (Ct) or other equivalent value at different nucleic acid concentrations and establishing a linear numerical relationship between Ct or equivalent value and apoptotic nucleic acid concentration; (d) subjecting test nucleic acid to real-time apoptosis-specific multi-product PCR in the presence of a nucleic acid-binding fluorophore or other detectable tag to obtain a Ct or other equivalent value; and (e) establishing the quantity of apoptotic nucleic acid in the test nucleic acid, which corresponds numerically to the concentration of nucleic acid in (c) which determines the Ct or equivalent value obtained in (d). A standard composition of isolated genomic nucleic acid comprising substantially 100% apoptotic nucleic acid or comprising apoptotic nucleic acid as a predetermined proportion of an isolated nucleic acid sample. A kit comprising same.

### RELATED APPLICATIONS

This application claims the benefit of U.S. Provisional Application No. 61/350,419 filed Jun. 1, 2010, which is hereby expressly incorporated by reference in its entirety.

### REFERENCE TO SEQUENCE LISTING

The present application is filed along with a Sequence Listing in electronic format. The Sequence Listing is provided as a file entitled 11331081.TXT, created May 31, 2011, which is approximately 1.11 Kb in size. The information in the electronic format of the Sequence Listing is incorporated herein by reference in its entirety.

### BACKGROUND OF THE INVENTION

1. Field of the Invention

The present invention relates to quantitative polymerase chain reaction (PCR) methods suitable for diagnosis or prognosis or for monitoring nucleic acid fragmentation, such as occurs during apoptosis. More particularly, to methods and products for use in quantifying original levels of apoptotic nucleic acid such as in a biological sample, after nucleic acid amplification reactions. Further, methods for determining the quantity of apoptotic DNA per cell number are provided. Still further, control samples comprising a pre-determined quantity of apoptotic DNA are provided. The methods and products will find broad application in a range of therapeutic, diagnostic, research and development assays. The methods do not require viable cells, can detect sub-nanogram amounts of nucleic acid and are suitable for high through put.

2. Description of the Related Art

Specific nucleic acid breaks underlie many morphological changes in normal and damaged or infected cells. Apoptosis is a regulated process of cell destruction initiated within cells and carried out by cellular enzymes as part of normal physiological processes. Proper regulation of apoptosis in man is essential for embryogenesis, post-natal tissue homeostasis and normal aging. Dysregulated apoptosis has been associated with, for example, pathological conditions including carcinogenesis, myocardial ischemia, neurodegenerative diseases, Alzheimers disease and infections such as acquired immunodeficiency syndrome. During apoptosis, inter-nucleosomal regions of genomic nucleic acid which occur every 180 by to 200 by are subject to specific enzymatic digestion resulting in the production of blunt-ended inter-nucleosomal fragment populations of about 200 by and multiples thereof (oligonucleosomal-sized fragments). Upon gel electrophoresis, these fragments form a characteristic “ladder”. The fact that DNA fragmentation is now known as a terminal stage in the apoptotic process stamps it as a reliable indicator and an accepted hallmark of apoptosis.

However, the proportion of nucleic acid that is fragmented in a tissue (for example, a clinical specimen) is typically too low to be directly visualized on a gel, even though changes at these low levels can reflect significant pathologies. Staley et al. Cell Death and Differentiation 4: 66-75, 1997 used a form of ligation-mediated PCR (LM-PCR) to amplify and visualize apoptotic fragments from a range of tissues from various species. Specifically, they disclose that apoptotic nucleic acid fragments are primarily blunt-ended and they used bacteriophage T4 DNA ligase to ligate a blunt-ended partially double-stranded oligonucleotide linker to blunt-ended apoptotic double stranded nucleic acid fragments. After a fill-in reaction, a primer having a nucleotide sequence complementary to the filled-in sequence at both ends of the double-stranded nucleic acid fragment was used in a standard PCR reaction to exponentially amplify multiple nucleic acid fragments and visualized by ethidium bromide staining of gel-resolved oligonucleosomal fragments. Although some relative quantification between gel-resolved samples was contemplated, no absolute measure of the amount of apoptotic nucleic acid was disclosed. There is currently no method for measuring in absolute terms the amount of apoptotic DNA from cells, and hence no method for using this value as a reliable marker for the extent of apoptosis.

Hooker et al., J. Cell. Mol. Med. 13(5): 948-958, 2009 describe a comparative quantitation method for assessing LM-PCR -mediated amplification of apoptotic nucleic acid by estimating the intensity of ethidium bromide staining of amplified 600 by oligonucleosomal-sized fragments after resolution by agarose gel electrophoresis.

Other methods for detecting apoptosis include TUNEL (Terminal deoxy nucleotidyl transferase-mediated dUTP nick end labeling) which uses a terminal transferase enzyme (TdT) to incorporate labeled nucleotides (dNTPs) into nucleic acid fragments containing a free 3′-hydroxyl group. Another approach uses the Klenow fragment of DNA polymerase I to label stretches of single stranded nucleic acid (gaps) and various 5′ overhangs present in various forms of nucleic acid breaks. The label may be detected using a variety of methods including immunochemical or fluorescent-based methods. Notably, these methods are cell based and are not specific to apoptotic or blunt-ended DNA. For example, TdT can extend the 3′ base of not only blunt-ended DNA but also single-strand breaks, overhangs and recessed 3′ bases of double stranded DNA.

Cysteine proteinases of the caspase family are activated during apoptosis and mediate, inter alia, degradation of proteinaceous cellular components. Various kits are available for monitoring caspase activity, such as the activity of caspase 3 or ICE, in enzyme preparations from cellular samples. In an illustrative example, enzyme substrates are labeled with a fluorophore and cleavage by enzyme releases the fluorophore for subsequent quantification of enzyme activity.

Methods of detecting apoptosis are described in detail in Roche Applied Science: Apoptosis, Cell Death and Cell Proliferation Manual, 3rd Edition, Roche Diagnostics GmbH available on line and incorporated herein by reference.

Quantitative PCR, refers to PCR which involves determining the absolute or relative amount of starting target material based on the amount of target product produced during amplification. It is technically challenging and has been considered as something of a black art. Illustrative methods are described in “Introduction to Quantitative PCR Methods and Applications Guide IN 70200 B, Copyright 2007 by Stratagene.

Real-time (qPCR) is a widely used method of quantitative PCR, typically employing a fluorometer, that is widely used to quantitate differences in mRNA expression. Real-time PCR involves quantifying the amounts of reference and target sequences in consecutive cycles within the exponential phase of PCR. Amplified products accumulate during the exponential phase of PCR according to the following equation logN_{f}=n log (1+Y)+logN₀ where N_{f} is the copy number of the amplified sequence after n cycles at amplification, N₀ is the initial copy number of the target sequence in the nucleic acid template and Y is the efficiency of amplification per cycle. A semi-logarithmic graphical plot of log concentration of a particular amplified product against cycle number yields a straight line. The equation describing the accumulation of the product may be derived using regression analysis. The quantity of the starting test sequence may be determined by simple interpolation into a standard curve and/or regression analysis of the accumulation of the amplification products during successive cycles of PCR. Quantifying the amplified nucleic acid using data gained during the exponential phase of PCR, when none of the components of the reaction are limiting, provides improved results. However, it is considered that accurate quantitative determinations in real-time PCR requires a single species of amplification product. In the case of LM-PCR of oligonucleosomal-sized fragments, multiple different fragment lengths and multiple different nucleotide sequences are amplified suggesting that it would be unsuitable for real-time PCR.

Fluorometric detection of PCR products is particularly convenient as it is highly sensitive and permits measurement of quantities of amplified nucleic acid over a range of at least six or seven orders of magnitude. One form of fluorometric detection uses generic nucleic acid binding dyes such as ethidium bromide and SYBR Green I. These are “universal dyes” that detect double-stranded nucleic acid generated during PCR. As many molecules of the dye combine with each double-stranded nucleic acid product, the intensity of signal generated is high and is proportional to the total mass of double-stranded nucleic acid generated during the PCR. Non-specific amplification, or the formation of primer dimers, can generate significant errors in quantifying small numbers of target molecules when universal dyes are used as markers.

There is a need in the art for improved methods for quantifying nucleic acid fragmentation, including nucleic acid fragmentation associated with apoptosis.

### SUMMARY OF THE INVENTION

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided after the claims.

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Each embodiment described herein is to be applied mutatis mutandis to each any every embodiment unless specifically stated otherwise.

The present invention provides, inter alia, methods for determining the quantity of apoptotic nucleic acid in a biological or nucleic acid sample.

The present invention provides methods for quantifying apoptosis using real-time nucleic acid amplification technology to quantify the components of multi-product ligation-mediated nucleic acid amplification. By “multi-product” is meant a plurality of amplification products that differ by length (mass) and/or composition, which are amplified using a single primer or a single primer pair. In the example of apoptotic genomic DNA comprising nucleic acids of about 200 by and multiples thereof, each population of approximately the same length comprises a plurality of nucleotide sequences that may differ substantially in composition.

Reference to multiple products means a plurality of different products. Products are not, for example, allele specific or naturally or unnaturally occurring variants of single nucleotide sequences. The plurality of products typically includes those having less than 60%, typically less than about 50%, less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.1%, sequence identity, excluding the primer target sequence.

Control DNA comprising a known amount of apoptotic nucleic acid may be assessed separately and/or at the same time as assaying test nucleic acids and may be obtained from the same or a different source. In some embodiments, the methods comprise: (a) obtaining first control genomic nucleic acid which is genomic nucleic acid derived from a cellular sample that comprises a pre-determined amount of substantially 100% apoptotic nucleic acid.

In other some embodiments, the methods comprise: (a) obtaining first control genomic nucleic acid which is genomic nucleic acid derived from a cellular sample wherein the nucleic acid is substantially 100% apoptotic.

In further embodiments, the methods comprise (b) subjecting a plurality of pre-determined quantities of first control genomic nucleic acid to real-time multi-product apoptosis-specific nucleic acid amplification, such as PCR. In some embodiments, (b) is conducted in the presence of a nucleic acid-binding dye or other detectable tag to obtain a threshold cycle number (Ct) or other equivalent value so as to establish a linear numerical relationship between Ct or equivalent value and nucleic acid quantity. In some embodiments, nucleic acid amplification is PCR. In some embodiments, PCR is LM-PCR wherein linkers are joined to the ends of oligonucleosomal-sized nucleic acid fragments to provide a substrate for a primer for nucleic acid amplification.

In some embodiments, the method further comprises (c) obtaining test genomic nucleic acid derived from a test cellular sample.

In further embodiments, the method comprises (d) subjecting test genomic nucleic acid to real-time multi-product apoptosis-specific PCR in the presence of a nucleic acid-binding dye or other detection tag to obtain a Ct or other equivalent value. In some embodiments, apoptosis-specific PCR is LM-PCR.

In a further embodiment, the methods comprise (e) establishing the quantity of apoptotic nucleic acid in the test genomic nucleic acid, which corresponds numerically to the quantity of nucleic acid in (b) which determined the Ct or equivalent value obtained in (d).

In another embodiment, the methods comprise: (f) obtaining second control genomic nucleic acid derived from a cellular sample of known cell numbers; (g) subjecting a plurality of pre-determined quantities of second control genomic nucleic acid corresponding to different cell numbers to real-time gene copy-number specific PCR in the presence of a nucleic acid-binding dye or other detectable tag to obtain a threshold cycle number (Ct) or other equivalent value and establishing there from a linear numerical relationship between Ct or equivalent value and cell number; (h) subjecting test nucleic acid to the real-time gene copy-number specific PCR in presence of a nucleic acid-binding dye or other detectable tag to obtain a Ct or other equivalent value; and (i) establishing the cell number corresponding to the test nucleic acid which corresponds numerically to the number of cells in (g) determining the Ct or equivalent value obtained in (h). In some embodiments, the method (f) to (h) (Cell Number qPCR) is conducted independent of steps (a) to (e). In this embodiment, the test nucleic acid and the second control nucleic acid may be derived from the same cell source.

Conveniently, in some embodiments the cell is a primary cell or a cell of known single copy number for the amplified region/gene.

In some embodiments, nucleic acid is amplified with a primer/primers which specifically amplify a single copy gene under the assay conditions employed.

Accordingly, in the above disclosed embodiments, either or both of the quantity of apoptotic nucleic acid and the cell number can be determined as a function of Ct or other equivalent value.

In further embodiments, the present invention provides substantially 100% purified apoptotic nucleic acid and a composition comprising same or 100% purified apoptotic nucleic acid produced according to the protocol established herein. These compositions are for use as standards in the subject real-time PCR assays as described herein. In some embodiments, the apoptotic DNA is purified by selecting nucleic acid in the range of 200 bp to 50 kbp.

In other embodiments, the present invention provides apoptotic nucleic acid as a predetermined proportion of a nucleic acid sample.

In some embodiments, apoptotic nucleic acid is sold together with instructions for use, or as part of a kit, such as a kit sold with instructions for use in the methods of the present invention. In some embodiments, the kits comprise a second control nucleic acid as defined herein.

In some embodiments, 100% apoptotic nucleic acid or another pre-determined percent apoptotic nucleic acid is provided in a range of several different quantities in separate containers ready for use for example as standard in the methods of the present invention.

The above summary is not and should not be seen in any way as an exhaustive recitation of all embodiments of the present invention.

### BRIEF DESCRIPTION OF THE DRAWINGS

FIG. 1 is a schematic diagram illustrating the molecular process of ligation-mediated PCR (LM-PCR) of apoptotic target nucleic acid (diagram and process modified from Staley et al., 1997 (supra)).

FIG. 2(A) is a photographical representation of gel electrophoresis showing production of 100% apoptotic nucleic acid for first control nucleic acid (for use in qLM-PCR standard curve).

FIG. 2(B) is a graphical representation of gel trace analyses of non apoptotic and apoptotic nucleic acid validating the definition of 100% apoptotic nucleic acid.

FIG. 2(C) is a graphical representation of trace intensities illustrating that the generation of substantially 100% apoptotic control samples is achievable and is reproducible.

FIG. 3 provides graphical representations of TUNEL/FACS analyses conducted to validate the definition of 100% apoptotic nucleic acid as described in Example 2.

FIG. 4 provides graphical representations of construction of a standard curve from substantially 100% apoptotic nucleic acid showing successful production of a substantially linear plot of apoptotic DNA on a logarithmic scale against mean of triplicate Ct values (qLM-PCR standard curve from first control nucleic acid), over a 1000-fold dynamic range.

FIG. 5 is a graphical representation illustrating the amplification of oligonucleosomal-sized nucleic acid fragments at different dilutions (qLM-PCR standard curve-amplification plots).

FIG. 6 is a photographic representation of resolved amplified fragments and size markers on an agarose gel showing that the products of amplification are oligonucleosomal-sized nucleic acid fragments (amplification products at 20 cycles).

FIG. 7 is a graphical representation showing a linear relationship between log cell number and mean Ct (Cell Number qPCR standard curve from second control nucleic acid).

FIG. 8 are graphical representations showing Jurkat cells or PBMC incubated with 0, 0.1, 0.5 μM staurosporine over 5 hours tested for apoptosis at 0, 1, 2, 3, 4 and 5 hour(s) by TUNEL/FACS, caspase 3 activity/ELISA or the present method.

### DETAILED DESCRIPTION OF THE PREFERRED EMBODIMENT

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

The articles “a” and “an” are used herein to refer to one or to more than one (i.e. to at least one) of the grammatical object of the article. By way of example, “an element” means one element or more than one element.

Functionally equivalent methods and products, and kits employing such methods and products are clearly within the scope of the invention as described herein.

The present invention is predicated, in part, upon the surprising development by the inventor of a combined ligation-mediated and real-time PCR method employing PCR amplification of multiple oligonucleosomal-sized fragments with a primer that binds to a linker region shared by each blunt-ended oligonucleosomal fragment. As known in the art, “real time” refers to methods wherein the products of amplification are assessed at various time points during the amplification protocol.

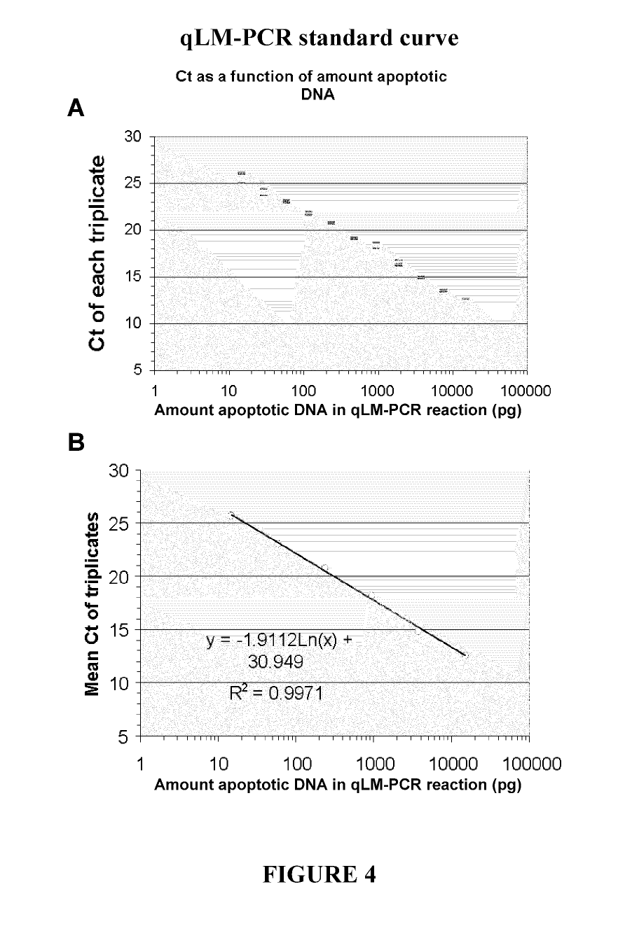

As shown in FIG. 4, the relationship between Ct and apoptotic DNA input amount into real-time PCR was found to be linear over a 1000-fold dynamic range. This was unexpected because accurate quantitative determination in real-time PCR are thought to require a single species of amplification product. The present real-time amplification methods permit the accurate determination of an absolute quantity of apoptotic nucleic acid in a sample of starting material based upon the quantity of amplified product produced over time. The method is readily adapted to high throughput analyses such as for testing clinical samples or for drug or other compound assays. It is capable of quantifying apoptosis over a range of apoptotic nucleic acid concentrations likely to encompass clinically relevant levels of apoptotic nucleic acid. Furthermore, the present method provides an essentially absolute quantification of apoptosis expressed as a measure of the quantity of apoptotic DNA/cell number such as picogram apoptotic DNA/10³ cells.

Reference to the phrase “derived from” or “obtained from” is meant that a sample such as, for example, a nucleic acid extract is isolated from, or originated from, a particular source. For example, the extract can be obtained from a tissue or a biological fluid isolated directly from a subject.

In one embodiment, the present invention provides a method for quantifying apoptosis by real-time ligation-mediated (LM) polymerase chain reaction (PCR), the method comprising: (a) obtaining first control genomic nucleic acid which is genomic nucleic acid derived from a cellular sample that is substantially 100% apoptotic; (b) subjecting a plurality of pre-determined quantities of first control genomic nucleic acid to real-time apoptosis-specific multi-product LM-PCR (“qLM-PCR”) in the presence of a DNA-binding dye or other detectable tag to obtain a threshold cycle number (Ct) or other equivalent value at different nucleic acid quantities and establishing a linear numerical relationship between Ct or equivalent value and nucleic acid quantity; (c) obtaining test genomic nucleic acid derived from a test cellular sample; (d) subjecting test genomic nucleic acid to real-time multi-product apoptosis-specific PCR (herein referred to as qLM-PCR) in the presence of a nucleic acid-binding dye or other detection tag to obtain a Ct or other equivalent value; and (e) establishing the quantity of apoptotic nucleic acid in the test genomic nucleic acid, which corresponds numerically to the quantity of nucleic acid in (b) which determines the Ct or equivalent value obtained in (d).

Cellular tissues generally comprise a small and unknown proportion of cells undergoing apoptosis. It has been estimated that tissue regression, for example, can take place as a result of only 3% of cells undergoing apoptosis. Within any cell undergoing apoptosis the proportion of nucleic acid that is fragmented is unknown. As a first step in determining this unknown quantity, and in accordance with the present invention, the inventor has produced a first control genomic nucleic acid sample that is substantially 100% apoptotic.

In an illustrative embodiment, Jurkat cells, an immortal human T-cell line, were incubated for various periods in the presence of different concentrations of an apoptosis inducer, the kinase inhibitor staurosporine. The amount of unfragmented genomic nucleic acid in the sample was determined by trace intensity analysis after gel electrophoretic resolution of isolated genomic DNA. As described in the Examples, under optimal conditions unfragmented nucleic acid was not detected in the test samples as determined by trace intensity analysis of electrophoresed samples. In a separate experiment this was repeated and was achieved again, and was therefore reproducible. To verify the percentage of apoptotic cells under optimal test conditions, the degree of apoptosis was also determined by terminal deoxynucleotidyl transferase-mediated dUTP nick end labeling (TUNEL) with flow cytometry of apoptosed cells (FACS) revealing 99% apoptotic cells.

Reference herein to an “apoptotic” cell or tissue is a reference to a cell or tissue that is or comprises cells that are undergoing apoptosis. Apoptotic cells comprise at least some oligonucleosomal-sized fragments indicative that apoptosis has been initiated in the cell.

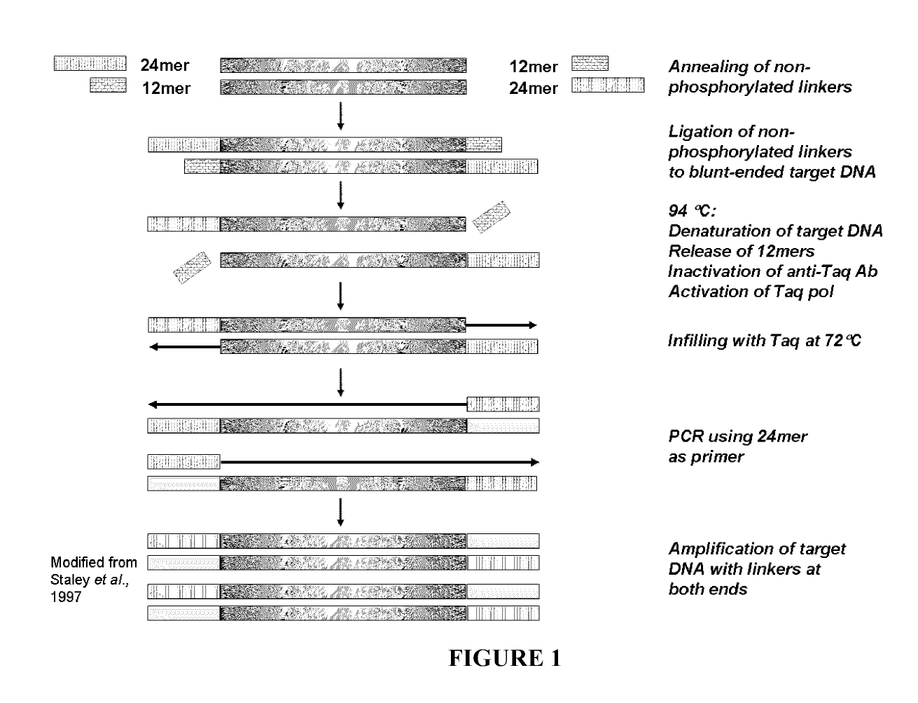

Reference herein to “apoptotic nucleic acid” means nucleic acid that has been subject to enzymatic activity that is a characteristic for apoptosis and is substantially in the form of apoptosis-specific oligonucleosomal-sized fragments and essentially lacks unfragmented nucleic acid. Apoptotic nucleic acid may form a proportion of the nucleic acid in a nucleic acid sample such as a sample comprising a proportion of cells undergoing apoptosis. Apoptotic-specific nucleic acid amplification means amplification that selectively amplifies apoptotic nucleic acid. In an illustrative embodiment, apoptotic-specific nucleic acid amplification comprises annealing or ligating a linker to blunt ends of nucleic acid oligonucleosomal-sized fragments and amplifying at least a proportion of so linked fragments using a primer that hybridise to a linker region of the ligated nucleic acid. In one illustrative embodiment, the method is essentially as set out in FIG. 1.

Reference herein to “100% apoptotic” refers to a control nucleic acid sample wherein all of the genomic nucleic acid present in the sample is present as oligonucleosomal-sized fragments and single approximately 200 by populations as determined by procedures such as disclosed herein.

Reference to “substantially 100% apoptotic” refers to a control nucleic acid sample or standard wherein all of the genomic nucleic acid present in the sample is present as oligonucleosomal-sized fragments as determined by standard procedures such as disclosed herein. The term further includes a nucleic acid sample wherein the proportion of nucleic acid in a sample which is present as oligonucleosomal-sized fragments is at least about 95% to 100%. Such a range includes 96%, 97%, 98%, 99% and 100% oligonucleosomally fragmented nucleic acid. In another embodiment, samples comprise less than 5% unfragmented genomic nucleic acid preferably less than 4%, 3%, 2%, 1.5%, 1.8%, 1.9% unfragmented nucleic acid. In one embodiment, percentages may be determined by ethidium bromide or SYBR Safe staining of electrophoretically resolved nucleic acid and by comparing trace intensities between fragmented and unfragmented nucleic acid. Typically, test and control apoptotic nucleic acid is blunt-ended, that is, wherein both strands of double stranded nucleic acid terminate in a base pair rather than some form of overhang.

Substantially 100% apoptotic nucleic acid provides a useful reagent that can readily be quantified, stored and reproduced. The present method uses isolated nucleic acid obviating the repeated need for viable cells or active enzymatic preparations. Putative apoptotic nucleic acid may be prepared (isolated) using methods described herein that are known in the art, from a range of tissues or cell types including haematopoietic cells, stem cells, immune cells, transformed cells, nerve cells, adipocytes, muscle cells, osteocytes, chondrocytes, epidermal cells, mucosal cells, endometrial cells and their precursors etc.

In other embodiments, first control nucleic acid comprises a pre-determined proportion of apoptotic nucleic acids present in the sample as oligonucleosomal-sized fragments or a known proportion of unfragmented nucleic acid. Thus, in some embodiments first control nucleic acid comprises one or more of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.8% apoptotic nucleic acid.

In some embodiments, the present invention provides a method of producing substantially 100% apoptotic nucleic acid. In an illustrative embodiment, the method comprises incubating a cellular sample with staurosporine or other apoptosis-inducing agent for a time and under conditions sufficient to induce apoptotic nucleic acid and isolating or purifying said nucleic acid. Optimal conditions include incubation for 5 hours at 37° C. with 8 μM staurosporine.

In some embodiments, the present invention provides substantially 100% apoptotic nucleic acid, such as, for example, genomic DNA produced by the above method.

In other embodiments, the present invention provides apoptotic DNA as a predetermined proportion of a nucleic acid sample. Thus 100% apoptotic DNA may be diluted with non-apoptotic nucleic acids.

In some embodiments, total apoptotic nucleic acid is sold together with instructions for use, or as part of a kit, such as a kit sold with instructions for use for example in the methods of the present invention.

In some embodiments, 100% apoptotic nucleic acid is provided in a range of several different quantities in separate containers ready for use in the methods of the present invention. Illustrative quantities would range from pg to ng quantities, from about 2 pg to 200 ng. In further embodiments, kits of the present invention may comprise second control nucleic acid to facilitate quantification relative to cell numbers, as described further below.

Kits contemplated include those designed to facilitate qLM-PCR, Cell Number qPCR and the combined method of ApoqPCR.

In some embodiments, kits suitable for qLM-PCR ApoqPCR comprise first control nucleic acid, such as a quantity of substantially 100% apoptotic nucleic acid. In this embodiment, kits further comprise a linker suitable for ligation to the ends of apoptotic oligonucleosomal-sized nucleic acid fragments. Suitable linkers include those that ligate efficiently to blunt-ended nucleic acid fragments. Such linkers are typically non-phosphorylated. In particular embodiments of ApoqPCR, kits further comprise a second control nucleic acid such as a quantity of nucleic acid from a suitable cell representing a known number of cells as described, for example, in the Examples. In this embodiment, kits also comprise a primer or primer pair suitable for amplifying a single copy gene from second control nucleic acid. Illustrative linker/linkers comprise a nucleic acid sequence as set forth in SEQ ID NO: 1 and SEQ ID NO: 2 or functional variants thereof. Kits for Cell Number qPCR comprise, inter alia, second control DNA as a standard for determining cell numbers in test DNA. Functional variants of linker sequences SEQ ID NO: 1 and/or SEQ ID NO: 2 are readily prepared and tested using standard procedures and as described herein. Generally, variants will differ from SEQ ID NO: 1 or 2 by as little as 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or up to about 50% sequence identity. However, linkers of any sequence may be employed provided that they are functionally equivalent in the present methods. Suitable alternative linker/primer sequences are designed and tested using routine methods together with the method provided herein.

The design of linkers/primers is suitably facilitated with the assistance of a computer programmed with software available in the art which searches a nucleotide sequence database comprising sequences of a plurality of genomes for sequences that will hybridize to linker sequences, but not to subsequences within the genomes or will not amplify PCR products using a pair of primers designed to hybridize to the sequence or its complement. The probability of a subsequence occurring in a polynucleotide is approximately described by P=1−(4^{X}/L) where P is the probability, L is the length of the polynucleotide and X is the length of the subsequence. The sequence is only approximate as it is affected by the biological nature of the sequences. As the length of the subsequence increases it becomes less and less common and oligonucleotides that match those subsequences become more and more specific. For example, a subsequence of 4 bases will occur in most polynucleotides of a few hundred bases whereas a 15-base subsequence would be expected to occur probably only once in a one billion base pair polynucleotide. Thus, a selection of linkers of approximately 15 to 30 or 12 to 20 basepairs that are specifically amplifiable within a human genome should be readily identified by this approach.

The kits of the present invention comprise one or more of a control nucleic acid (corresponding to first and/or second control nucleic acid), a linker, a primer, a non-specific annealing inhibitor, an amplification enzyme (such as a DNA polymerase), optionally a detection tag and optionally a reagent for detecting same, and optionally instructions for use. In some embodiments, the primer is detectably modified.

In still further embodiments described herein below, the kits of the present invention comprise one or more of a DNA ligase, a ligase buffer, one or more control nucleic acids, a linker, a primer or a primer pair, a non-specific annealing inhibitor, an amplification enzyme with buffers, deoxynucleotide triphosphates, MgCl₂, a detection tag and optionally a reagent for detecting same, and optionally instructions for use. In some embodiments, the primer is detectably modified.

In some embodiments, the non-specific annealing inhibitor is an anti-Taq monoclonal antibody.

In some embodiments, the present invention provides a kit for determining the quantity of apoptotic nucleic acid in a sample, wherein the kit comprises: (a) one or more portions comprising substantially 100% apoptotic first control nucleic acid; (b) a portion comprising a linker and a portion comprising a primer for LM-PCR of apoptotic nucleic acid; (c) optionally one or more portions comprising one of a detectable dye or tag, a ligase, a nucleic acid polymerase, a non-specific annealing inhibitor, a buffer, and dNTPs; and (d) instructions for using the components of the kit to determine the quantity of apoptotic nucleic acid in a test sample.

In some embodiments, the kit further comprises one or more portions comprising a second control nucleic acid.

Once a standard curve is established using the information obtained in (b) above, test nucleic acid can be tested following at least step (d) above.

In some embodiments, the present method uses real-time apoptosis-specific multi-product LM-PCR in the presence of a nucleic acid-binding dye or other detection tag to obtain a threshold cycle number (Ct) or other equivalent value at different nucleic acid quantities and establishing a linear numerical relationship between Ct or equivalent value and nucleic acid quantity.

In an illustrative embodiment, a modification of the method of Staley et al., (supra) is used for LM-PCR as described in the Examples. In particular, unphosphorylated oligonucleotides of 12 (short) and 24 (long) nucleotides are annealed to form a blunt ended partially double stranded linker. The linker is ligated to the blunt ends of double stranded oligonucleosomal fragments by bacteriophage T4 DNA ligase. The short oligonucleotide is removed (melted off) and the ends subjected to a fill-in reaction. A primer that hybridizes to the filled-in region at both ends of the fragment is used to amplify linked oligonucleosomal-sized fragments in a standard PCR reaction. In the illustrative examples, a single primer is employed. In some embodiments, a single primer pair or a small number of primers or primer pairs may be employed. In each case, the primer or primer pair amplifies multiple different products, i.e. of different length and/or composition.

In an illustrative, non-essential embodiment, quantification of the amplified product is achieved by incorporating the nucleic-acid binding dye, SYBR Green I into the amplification product. The use of fluorochromes such as SYBR Green as a detection tag is convenient. However, as the skilled person will appreciate, a large range of other detection tags known in the art can be employed in the present method. As will also be appreciated in the art, the detection (detectable) tag may, for example, be incorporated in the PCR primer or a separate oligonucleotide probe or the detection tag may be a universal tag that binds to double stranded DNA. Any convenient protocol and device for quantifying amplified product is encompassed. Currently available real-time PCR instrumentation conveniently automates measurements of fluorescence detection tags during the exponential phase of amplification in every PCR cycle and typically comprises software able to automatically perform data analyses.

In some embodiments, in step (c), first control nucleic acid in a range of pre-determined quantities is subjected to real-time qLM-PCR as described above and a threshold cycle number (Ct) is established. The threshold cycle number is the number of the PCR cycles at which the fluorescence signal measured by the PCR instrument reaches a pre-determined threshold value which is greater than background fluorescence levels and is measured within the exponential amplification phase of the PCR reaction. A standard curve or equation is generated defining the relationship between the log of quantity of starting material (first control nucleic acid) and the Ct. A linear relationship between log quantity and Ct indicates that the regression line can be used as a standard curve to interpolate the starting quantity of apoptotic nucleic acid in test samples from Ct values of test nucleic acid samples, where the quantity of apoptotic DNA is unknown.

In some embodiments, apoptotic nucleic acid is blunt-ended DNA.

In further embodiments, the method comprises (d) subjecting test genomic nucleic acid to real-time multi-product apoptosis-specific PCR in the presence of a nucleic acid-binding dye or other tag to obtain a Ct or other equivalent value. In an illustrative embodiment, the steps of (d) are essentially the same as for (c) except that test nucleic acid comprise an unknown proportion of apoptotic nucleic acid.

Nucleic acid preparation follows procedures as described herein and such as, without limitation, those set out in Sambrook et al., eds., Molecular Cloning. A Laboratory Manual, Cold Spring Harbor Press, 1989, Chapters 6 and 7. In preferred embodiments, DNA is isolated using commercially available spin column technologies that isolate DNA in the target size range of approximately of 200 by to 50 kbp.

Once a Ct value for the test nucleic acid has been established, the Ct value is used to determine the quantity of starting test apoptotic nucleic acid using the standard curve information obtained in (c). Thus (e) comprises establishing the quantity of apoptotic nucleic acid in the test genomic nucleic acid, which corresponds numerically to the quantity of nucleic acid in (c) which determines the Ct or equivalent value obtained in (d).

In a further embodiment, the method comprises: (f) obtaining second control genomic nucleic acid derived from a cellular sample of known cell numbers; (g) subjecting a plurality of pre-determined quantities of second control genomic nucleic acid corresponding to different cell numbers to real-time gene copy-number specific PCR in the presence of a nucleic acid-binding dye or other detection tag to obtain a threshold cycle number (Ct) or other equivalent value and establishing there from a linear numerical relationship between Ct or equivalent value and cell number; (h) subjecting test nucleic acid to the real-time gene copy-number specific PCR in presence of a nucleic acid-binding dye or other tag to obtain a Ct or other equivalent value; and (i) establishing the cell number corresponding to the test nucleic acid which corresponds numerically to the number of cells in (g) determining the Ct or equivalent value obtained in (h).

Conveniently, in some embodiments the cell is a primary cell or a cell of known or single copy number for the amplified region/gene.

In some embodiments, nucleic acid is amplified with a primer or primer pair specific for a single copy gene. In other embodiments, the copy number is a pre-determined number to facilitate comparative analyses.

Accordingly, in some embodiments, either or both of quantity of apoptotic nucleic acid and cell number can be determined as a function of Ct or other equivalent value. In some embodiments, when the methods employ both the method of qLM-PCR as described herein and the method of Cell Number qPCR as described herein, the method may be referred to as ApoqPCR.

The present invention refers to various “nucleic acids” such as those comprising gene sequences or a probe or primer. As used herein, a “nucleic acid” or “nucleic acid” or “nucleic acid molecule” means a polymer of nucleotides, which may be DNA or RNA or a combination thereof, and includes mRNA, cRNA, gDNA, cDNA, tRNA, siRNA, shRNA and hpRNA. Typically, the starting material in ApoqPCR, LM-PCR and Cell Number qPCR is genomic DNA. However, it may be DNA or RNA of cellular, genomic or synthetic origin, for example made on an automated synthesizer, and may be combined with carbohydrate, lipids, protein or other materials, labeled with fluorescent or other groups, or attached to a solid support to perform a particular activity defined herein. Nucleotides of the polymer may be modified according to methods known in the art, for example, analogs of phosphodiester linkages include: phosphorothioate, phosphorodithioate, phosphoroselenoate, phosphorodiselenoate, phosphoroanilothioate, phosphoranilidate, phosphoramidate, but the nucleic acids are preferably unmodified or modified only as occurs in a cell. The polymer may be single-stranded, essentially double-stranded or partly double-stranded. An example of a partly-double stranded RNA or DNA molecule is a hairpin RNA or DNA (hpRNA or hpDNA), short hairpin RNA (shRNA) or self-complementary RNA which include a double stranded stem formed by base pairing between a nucleotide sequence and its complement and a loop sequence which covalently joins the nucleotide sequence and its complement. Basepairing as used herein refers to standard base pairing between nucleotides, including G:U basepairs. “Complementary” means two nucleic acids are capable of base pairing along part of their lengths, or along the full length of one or both.

By “isolated” nucleic acid is meant material that is substantially or essentially free from components that normally accompany it in its native state. The present nucleic acids are isolated in this manner. Preferably, the isolated nucleic acid is at least 90% or essentially free from other components such as proteins, carbohydrates, lipids that may reduce reaction efficiencies.

The present invention refers to use of “linkers” “primers” and “probes”. As used herein, these molecules are “oligonucleotides” that are nucleic acids up to 50 nucleotides in length. They can be RNA, DNA, or combinations or derivatives of either. Oligonucleotides are typically relatively short single stranded molecules of 10 to 30 nucleotides, commonly 15-25 nucleotides in length. When used as a probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably about 25 nucleotides in length. Nucleic acids used as a probe are typically conjugated with a detectable label such as a radioisotope, an enzyme, biotin, a fluorescent molecule or a chemiluminescent molecule.

The term oligonucleotide/s as used herein refers to a polymer composed of a multiplicity of nucleotide residues (deoxyribonucleotides or ribonucleotides, or related structural variants or synthetic analogues thereof) linked via phosphodiester bonds, or related structural variants or synthetic analogues thereof, such as ‘locked nucleic acids’ (e.g., conformationally restricted nucleotide analogues with an extra 2′-O,4′-C-methylene bridge added to the ribose ring; Christensen et al., Biochem J 354: 481-484, 2001). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 8 to 30 nucleotides, more preferably from about 12 to 24 nucleotides and still more preferably from about 11 to 17 nucleotides, but the term can refer to molecules of any length, although the term “nucleic acid” or “nucleic acid” is typically used for large oligonucleotides. Oligonucleotides may be prepared using any suitable method, such as, for example, the phosphotriester method as described in an article by Narang et al., Methods Enzymol. 68: 90, 1979 and U.S. Pat. No. 4,356,270. Alternatively, the phosphodiester method as described in Brown et al., Methods Enzymol. 68: 109, 1979 may be used for such preparation. Automated embodiments of the above methods may also be used. For example, in one such automated embodiment, diethylphosphoramidites are used as starting materials and may be synthesized as described by Beaucage et al., Tetrahedron Letters 22: 1859-1862, 1981. Reference also may be made to U.S. Pat. Nos. 4,458,066 and 4,500,707, which refer to methods for synthesizing oligonucleotides on a modified solid support. It is also possible to use a primer, which has been isolated from a biological source (such as a denatured strand of a restriction endonuclease digest of plasmid or phage DNA). Oligonucleotides may be attachably modified. The term “attachably modified” is a broad reference to any modification made to an oligonucleotide in order to facilitate, directly or indirectly, its attachment to a substrate. For example, the oligonucleotide may be biotin labeled to facilitate attachment to bound streptavidin. Alternatively, the 5′ portion of the oligonucleotide may comprise sequences useful for binding to proteins or comprising a restriction site to facilitate ligation of an adaptor molecule capable of, for example attachment to a substrate.

In designing a primer for nucleic acid amplification consideration must of course be given to the selection of amplification reaction employed. The skilled person would understand the importance of considering the specificity of target amplification. The various parameters which can be optimized in order to ensure successful amplification reactions are well known and may be found described in general molecular laboratory manuals such as, for example, Ausubel et al., 1995 (supra), and Sambrook et al., 1989 (supra) as well as dedicated PCR manuals. Importantly, oligonucleotides may be modified to enhance both amplification success and detection of amplified products. For example the GC content of the oligonucleotide may be modified to alter the melting temperature. Universal nucleotide analogues such as for example, 5-nitroindole may be introduced into oligonucleotide sequences to enhance hybridization. Another design related option is to predict the composition of any mismatched hybridizations. Specifically, if a primer is likely to hybridize to a sequence of a non-target sequence the oligonucleotide is designed so that the resulting duplex includes at least one unmatched base close to the 3′ end of the primer to ensure that the oligonucleotide does not prime amplification.

Real-time PCR is conveniently carried out using a combined or coordinated thermocycler and fluorometer which can amplify specific nucleic acid sequences and measure their concentration simultaneously. The yield of amplified nucleic acid may be assessed using double stranded nucleic acid intercalating dyes such as SYBR Green 1. In this way the existence of an amplified product may be detected. In order to distinguish between amplified products, the melting temperature or electrophoretic mobility of amplified products may be assessed. The addition of non-target sequence nucleotides to the 5′ portion of an oligonucleotide primer may facilitate distinguishing amplified products, if required.

Labeled oligonucleotide probes may also be used to quantify amplified products in real-time amplification reactions. A number of different assay formats are available in which the signal from the label, usually a fluorescence label or dye, is detected after extension of an oligonucleotide probe by a polymerase having 5′-3′ exonuclease activity. For example, oligonucleotide probes are used bearing a fluorescent group at the 5′ end and a quenching molecule at the 3′ end. When both groups are close, the quencher quenches the signal from the fluorescence group. During the exponential phase of PCR, for example, the exonuclease activity of the polymerase cleaves the fluorescence label which, freed of the quencher emits a detectable signal in a fluorometer.

The cells and nucleic acids referred to herein may be derived from any organism. The term organism includes plants, animals (including humans), birds, insects, parasites, viruses, fungi, vertebrates, invertebrates etc.

Any detectable or attachable modification system may be employed such as those reviewed in Syvanen Anne-Christine, Nature Genetics, 2: 930-940, 2001 or described in Sambrook et al., 1989 (supra) (Appendix 9-Detection systems, Appendix 10-DNA array technology). For example, biotin or digoxigenin labeled oligonucleotides care attached to anti-biotin or anti-digoxigenin antibodies, respectively. Detection may be via conjugated enzyme systems such as horseradish peroxidase or alkaline phosphatase to generate a colour change reaction.

The detectable tag or modification is conveniently selected from a non target nucleic acid sequence, a chromogen, a catalyst, an enzyme, a dye such as an infrared dye, flurochrome, a chemiluminescent, bioluminescent or phosphorescent moiety, a lanthanide ion, a radioisotope or a visual label such as gold or silver nanoparticles. Fluorescent dyes are particularly well established however, this is a rapidly moving field and the present invention is in no way limited to the use of any particular detectable modification. In one particular embodiment, fluorophores, dyes or particles are employed.

In the case of a direct visual label, use may be made of a colloidal metallic or non-metallic particle, a dye particle, an enzyme or a substrate, an organic polymer, a latex particle, a liposome, or other vesicle containing a signal producing substance and the like. Especially preferred labels of this type include large colloids, for example, metal colloids such as those from gold, selenium, silver, tin and titanium oxide. In one embodiment in which an enzyme is used as a direct visual label, biotinylated bases are incorporated into a target nucleic acid. Hybridization may be detected, for example, by incubation with streptavidin-reporter molecules.

Suitable fluorophores include, but are not limited to, fluorescein isothiocyanate (FITC), tetramethylrhodamine isothiocyanate (TRITC), R-Phycoerythrin (RPE), and Texas Red. Other exemplary fluorophores include those discussed by Dower et al. (International Publication WO 93/06121). Reference also may be made to the fluorochromes described in U.S. Pat. No. 5,573,909 (Singer et al.), U.S. Pat. No. 5,326,692 (Brinkley et al.). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218. See also US Publication No. 20040216180 incorporated herein by reference. Commercially available fluorescent labels include, for example, fluorescein phosphoramidites such as Fluoreprime (Pharmacia), Fluoredite (Millipore) and FAM (Applied Biosystems International). Radioactive reporter molecules include, for example, ³²P, which can be detected by a X-ray or phosphoimager techniques.

Oligonucleotide probes or primers may be immobilized to a solid support using any suitable technique.

It will of course be appreciated that the oligonucleotides used in the invention may be immobilized either directly or indirectly. For example, a primer or probe may be adsorbed to a surface or alternatively covalently bound to a spacer molecule, which has been covalently bound to the solid support. The spacer molecule may include a latex microparticle, a protein such as bovine serum albumin (BSA) or a polymer such as dextran or poly-(ethylene glycol). Such a spacer molecule is considered to improve accessibility of the oligonucleotide primer to hybridization of the target nucleotide sequence. Alternatively, the spacer molecule may comprise a homo-nucleic acid tail such as, for example, oligo-dT. In a preferred embodiment, the spacer molecule is 10 to 25 molecules in length.

Suitable test cellular samples are any cell or unicellular organism. Suitable test samples include extracts of nucleic acid or copies therefrom from any organism. Extracts may be obtained from any source including cells or tissues or other biological material(s) including, without limitation, archive or ancient biological material derived from plants, parasite, fungi, bacteria, animal or viral sources.

Nucleic acid from test and control samples may be prepared by any suitable protocol. In this regard, reference may be made to the examples and to Laboratory Manuals for example Ausubel et al., 1995 (supra), and Sambrook et al., 1989 (supra). Sample extracts of RNA may be prepared by any suitable protocol as for example described in Ausubel et al., 1995 (supra), Sambrook et al., 1989 (supra) and Chomczynski and Sacchi, Anal. Biochem. 162: 156, 1987 hereby incorporated by reference.

Genomic DNA or RNA or cDNA may be fragmented to facilitate the analysis. Particular fragments may be enriched and unwanted fragments removed. Nucleic acids are amplified using suitable nucleic acid amplification techniques. Such amplification techniques are well known in the art and include for example PCR, Strand Displacement Amplification (SDA) (U.S. Pat. No. 5,422,252, Little et al.), Rolling circle Amplification (RCA) (Liu et al., J. Am. Chem. Soc 118: 1587-1594, 1996; International Publication no. WO 92/01813), Nucleic acid Sequence Based Amplification (NASBA) (Sooknanan et al. Biotechniques 17: 1077-1080, 1994) and Q-Beta replicase amplification (Tyagi et al., Proc. Natl. Acad. Sci USA 93: 5395-5400, 1996).

Amplification reactions employing one or more primers in accordance with the present invention are standard reactions whose management and optimization are well known to the skilled addressee. While the oligonucleotide primers are designed with their use in hybridization and amplification reactions in mind, nevertheless optimal conditions, including hybridization conditions for probe or primer binding, may be determined empirically using routine procedures. In this regard, reference may be made to the Examples and to Laboratory Manuals for example Ausubel et al., 1995 (supra), and Sambrook et al., 1989 (supra).

Homogeneous, solution phase amplification reactions are contemplated.

Alternatively, or in addition, various steps in the reactions may be carried out using solid supports, chromatography or electrophoresis to separate amplified products.

Depending on the nature of a detectable tag associated with an amplified product, a signal may be instrumentally detected by irradiating a fluorescent label with light and detecting fluorescence in a fluorometer; by providing for an enzyme system to produce a dye which could be detected using a spectrophotometer; or detection of a dye particle or a coloured colloidal metallic or non metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focused beam or laser light. In such a case, a charge couple device (CCD) or a photocell can be used to scan for emission of light from a probe:target nucleic acid/amplified product hybrid from each location in the micro-array and record the data directly in a digital computer. In some cases, electronic detection of the signal may not be necessary. For example, with enzymatically generated colour spots associated with nucleic acid array format, as herein described, visual examination of the array will allow interpretation of the pattern on the array.

In the case of a nucleic acid array, the detection means is preferably interfaced with pattern recognition software to convert the pattern of signals from the array into a plain language genetic profile.

The data from assessing any amplified products is compiled. The data is most conveniently analyzed with the use of a programmable digital computer. Computer methods for analyzing real time PCR data are taught in the prior art. In a preferred embodiment the programmable computer would contain specialist software code and register data.

The term “amplification product” refers herein to the product of polymerase or ligase mediated amplification reactions.

The phrase “detectably modified” refers broadly to any modification of an oligonucleotide or nucleic acid which facilitates its detection, either directly or indirectly. The phrase includes reference to detectable (detection) tags (labels/reporters).

By “gene” is meant a genomic nucleic acid sequence at a particular genetic locus.

By “pair” is meant two oligonucleotides comprising primer sequences (or one oligonucleotide comprising two primer sequences) which work together by hybridizing to and amplifying a target nucleic acid. The two oligonucleotides of a pair hybridize specifically to adjacent or spaced target sequences on the same or opposite strands. When used in polymerase dependent amplification reactions the pair typically define forward and reverse primers to amplify a product nucleic acid in a PCR reaction i.e., where an extension product synthesized from one hybridized oligonucleotide serves as a template for synthesis of an extension product of the other oligonucleotide. For ligation dependent reactions, adjacent members of the pair of hybridized oligonucleotide primers are ligated together and may form the substrate for hybridization of further similar pairs of oligonucleotides.

“Probe” refers to a single or double stranded oligonucleotide molecule that binds to a specific target sequence including a linker or primer sequence. The probe may bind to the products of nucleic acid amplification reactions designed to amplify a target sequence within the test nucleic acid. Probes can bind target nucleic acids or amplified products lacking complete sequence complementarity with the probe, depending on the stringency of the hybridization conditions. Oligonucleotide probes may be selected to be “substantially complementary” to a target sequence or adaptor (linker). The exact length of the oligonucleotide probe will depend on many factors including temperature and source of probe and use of the method.

The present invention is further described by the following non-limiting Examples.

### EXAMPLE 1

### The Generation of “100%” Apoptotic Nucleic Acid is Achievable and Reproducible

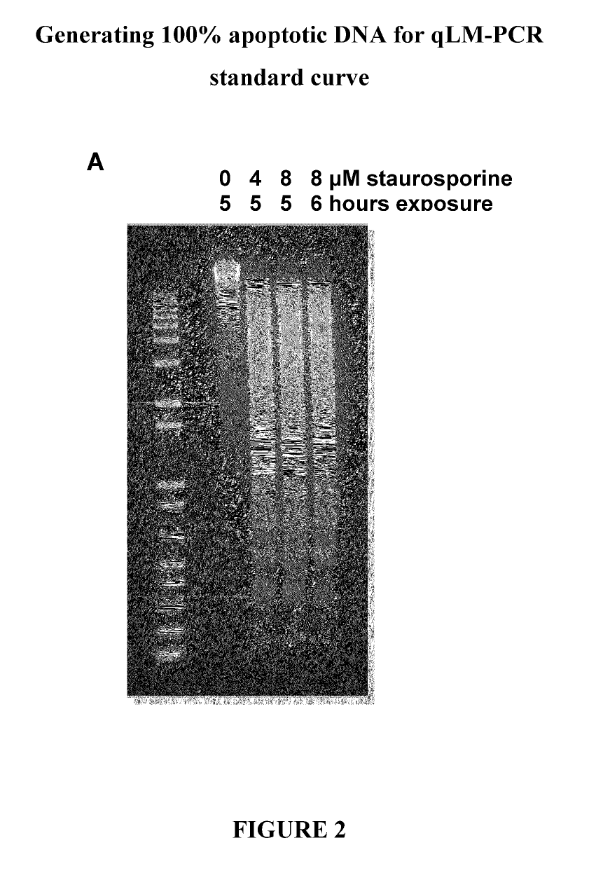

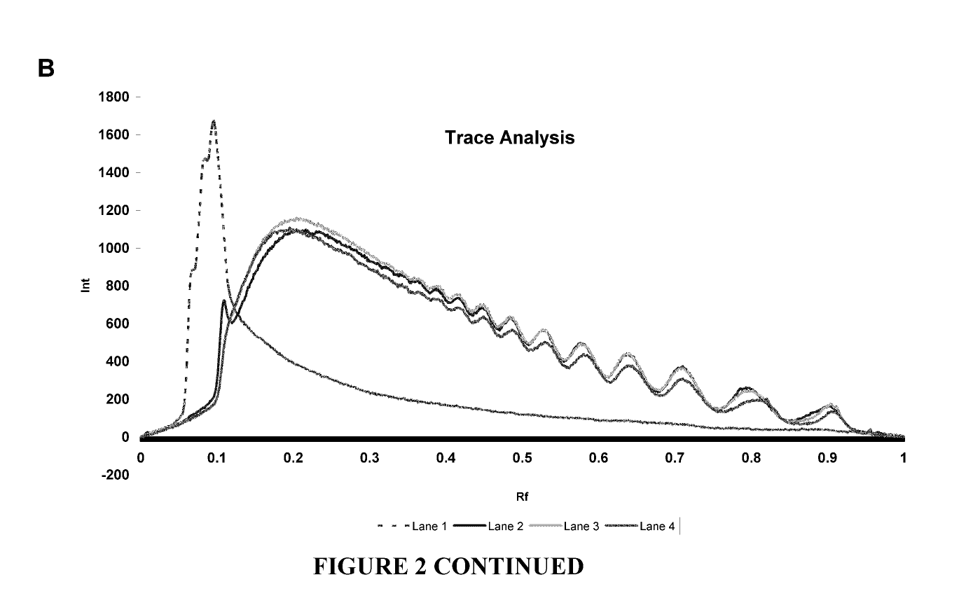

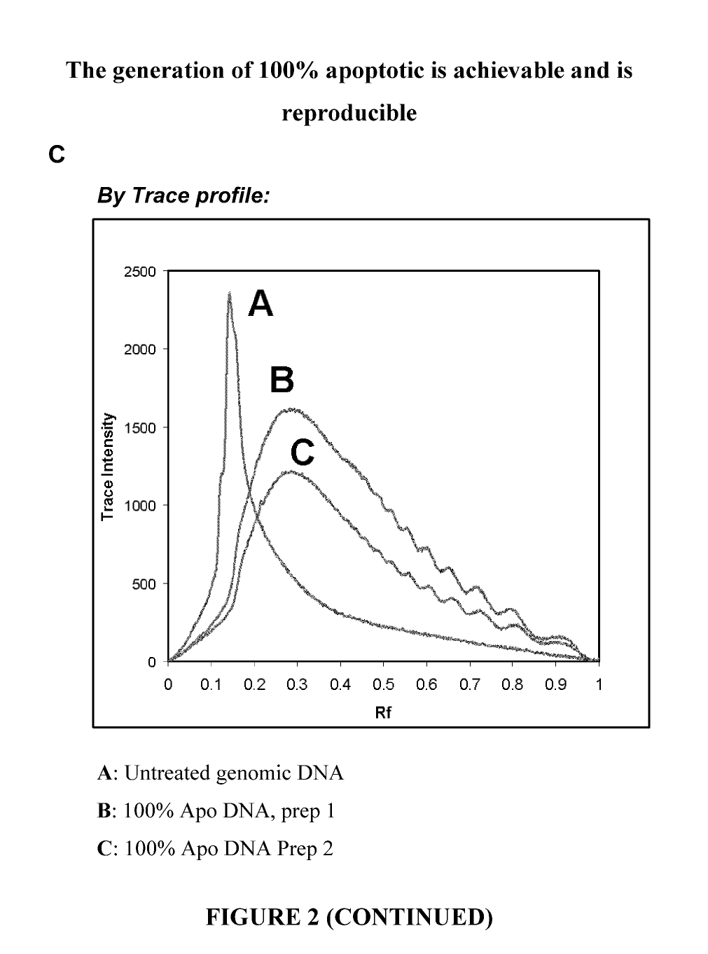

Jurkat cells were grown to log phase in RF-10 tissue culture medium, pelleted and resuspended to 10⁶ cells/ ml in new RF-10 at 37° C., and incubated at 37° C. with or without staurosporine (at 4 or 8 μM) for 5 and 6 hours. At the end-points, genomic DNA was purified using Qiagen DNA mini-prep columns following the manufacturer's recommendations, including RNase treatment and stored at −80° C. These columns quantitatively purify nucleic acid in the target size range of 200 by to 50 kbp. Nucleic acid for each end-point was compared by agarose gel electrophoresis. The generation of 100% apoptotic nucleic acid was defined as the complete absence of any typically high molecular weight genomic nucleic acid and complete conversion of this length of nucleic acid into apoptotic fragments, as determined by trace intensity using a GelDoc Imager (BioRad). Incubation with 4 μM staurosporine for 5 hours generated a nucleic acid population with 1.8% unfragmented high molecular weight genomic nucleic acid remaining as determined by trace quantification. The optimal conditions were found to be incubation for 5 hours at 37° C. with 8 μM staurosporine where all DNA was fragmented as determined by trace analysis (FIG. 2A, B). This was defined as 100% apoptotic DNA. The same experiment from cell culture to nucleic acid purification was repeated to test reproducibility. Nucleic acid from both experiments was electrophoresed together and trace intensities compared (FIG. 2C).

### EXAMPLE 2

### The Generation of 100% Apoptotic Nucleic Acid is Verifiable by Other Procedures for Quantifying Apoptosis

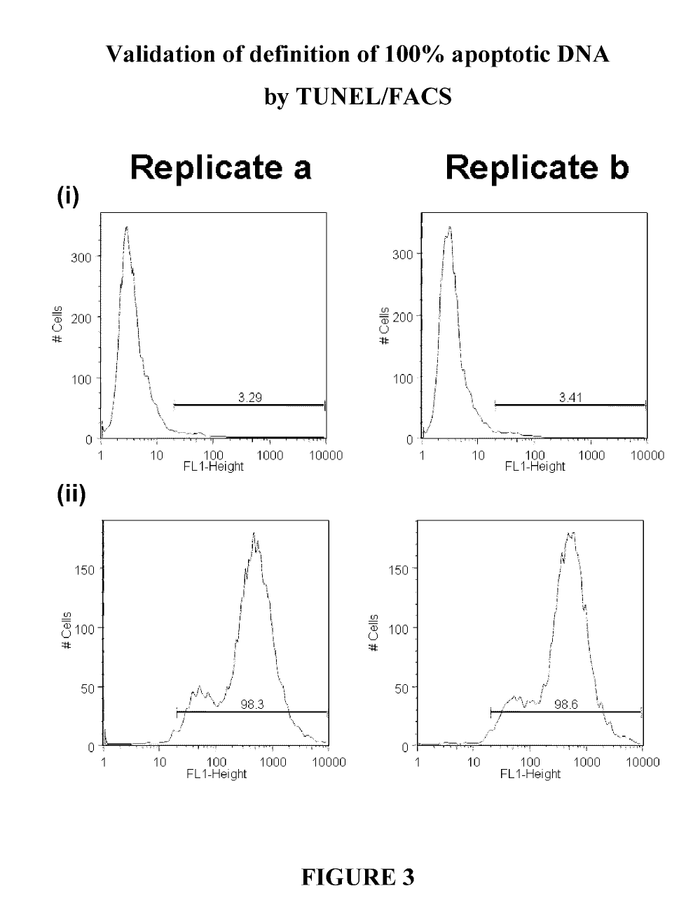

To verify the definition of 100% apoptotic DNA, apoptosis was measured by TUNEL with flow cytometry from fractions of the same cultured cells used for genomic nucleic acid purification (above). At each time point and each staurosporine concentration, cells (6 ml) were collected, washed twice with PBS and resuspended in PBS to 2×10⁷ cells/ml for duplicate 100 μl cells per micro-well. TUNEL procedure for cell suspensions followed the In Situ Cell Death Detection Kit, Flourescein (Roche, Mannheim, Germany) with the following modifications: after pelleting cells in wells, supernatant was aspirated and cells gently resuspended in 100 μl/well of fixation/permeabilisation solution (Cytofix/Cytoperm kit, BD Biosciences/Pharmingen, CA, USA). After incubation for 20 minutes at 4° C., cells were washed at this and subsequent wash stages 2 times at room temperature with perm/wash buffer (Cytofix/Cytoperm kit). After final washes, cells were resuspended in 250 μl/well 1% paraformaldehyde in PBS, stored at 4° C. in the dark for up to 12 hours and analyzed by flow cytometry at excitation 488 nm, detection 530 nm using a BD FACScan (BD Biosciences). At each measurement at least 10,000 events were sorted. Mean TUNEL positivity for cells incubated for 5 hours without staurosporine was 3%, while positivity for cells incubated for 5 hours with 8 μM staurosporine was 98.5% (FIG. 3).

### EXAMPLE 3

### Generation of Standard Curve Nucleic Acid for Apoptotic DNA Quantitation by Real-Time LM-PCR

100% apoptotic nucleic acid was quantitated spectrophotometrically, and a 2-fold or 4-fold dilution series constructed with T[E/2], (10 mM Tris-HCl pH 8.0 0.5 mM EDTA) such that 12 μl annealing/ligation reactions contain from 80 ng to 0.078125 ng apoptotic DNA, a 1000-fold dynamic range. After dilution of the completed annealing/ligation reactions to 40 μl with T[E/2] and subsequent addition of 7.5 μl to 25 μl qLM-PCR reactions, this equates to 15,000 pg to 14.64 pg in each 25 μl qLM-PCR reaction (Table 1).

Previous LM-PCR reactions not designed for real-time PCR (Staley et al. 1997 (supra)) routinely contained the equivalent of 200 ng genomic nucleic acid in 12 μl annealing/ligations. The range of diluted apoptotic nucleic acid for the standard curve in the current work equates to from 40% to 0.0390625% apoptotic DNA, a range covering clinically relevant levels.

### EXAMPLE 4

### Relationship Between Standard Curve Apoptotic DNA and Ct

A series of apoptotic DNA dilutions, constructed for the qLM-PCR standard curve, were made target DNA in qLM-PCR reactions. The relationship between Ct and apoptotic DNA input amount was found to be linear in a semi-logarithmic graphical plot over a 1000-fold dynamic range (FIG. 4).

### EXAMPLE 5

qLM-PCR Consistently Amplifies Oligonucleosomal-Sized Apoptotic Fragments Over a Range of Target Nucleic Acid Input Amounts

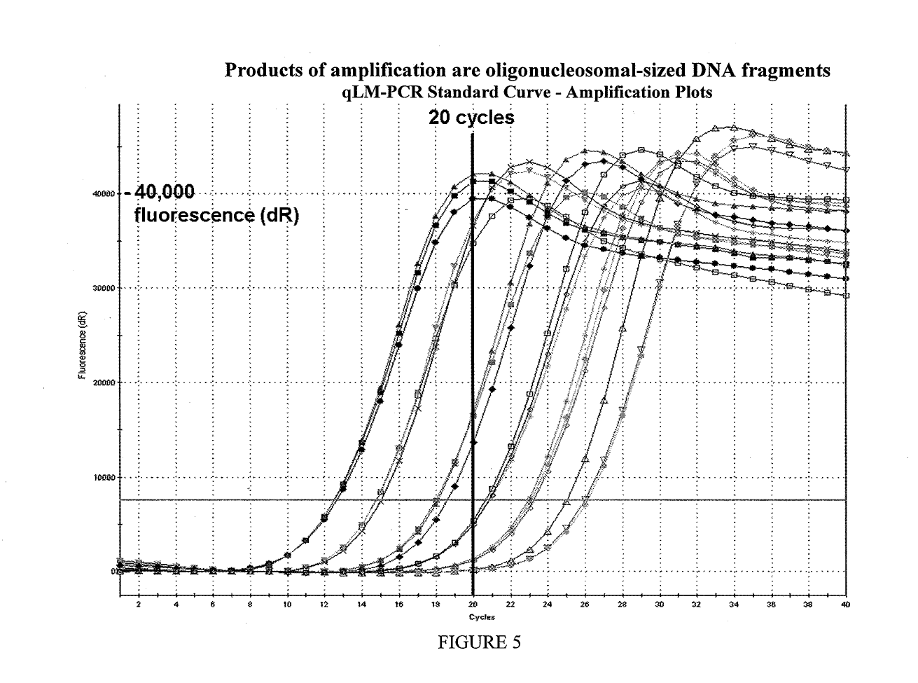

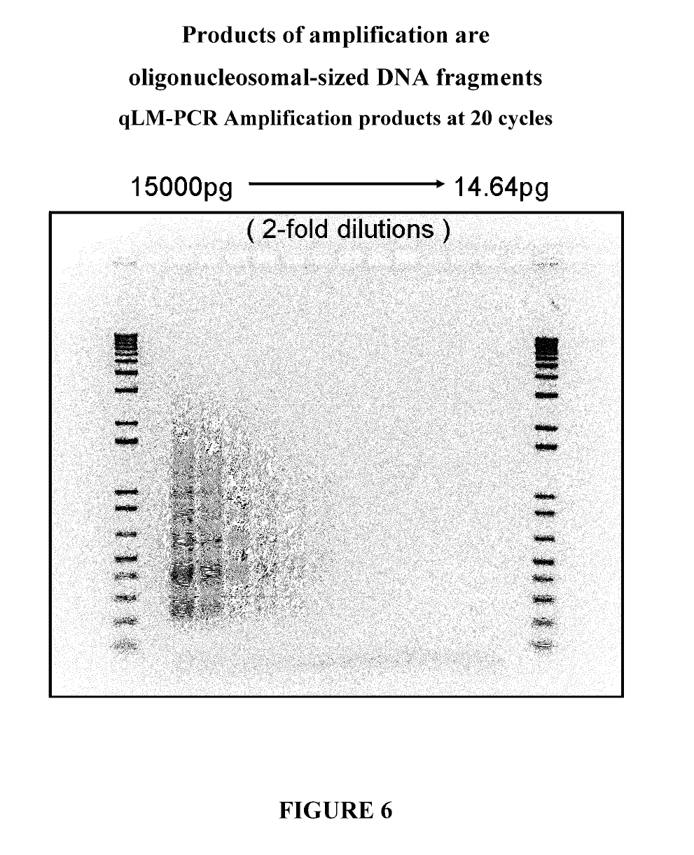

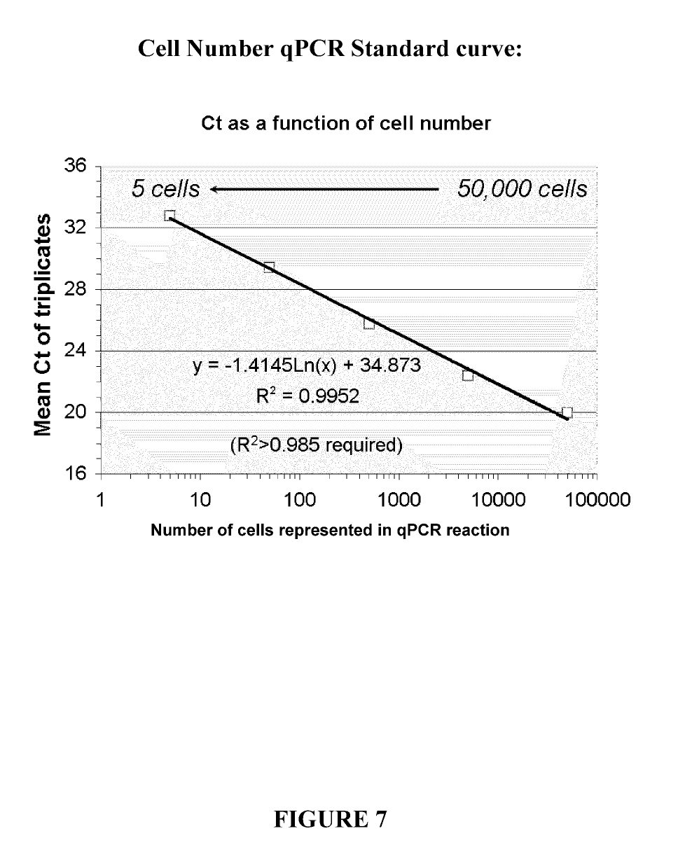

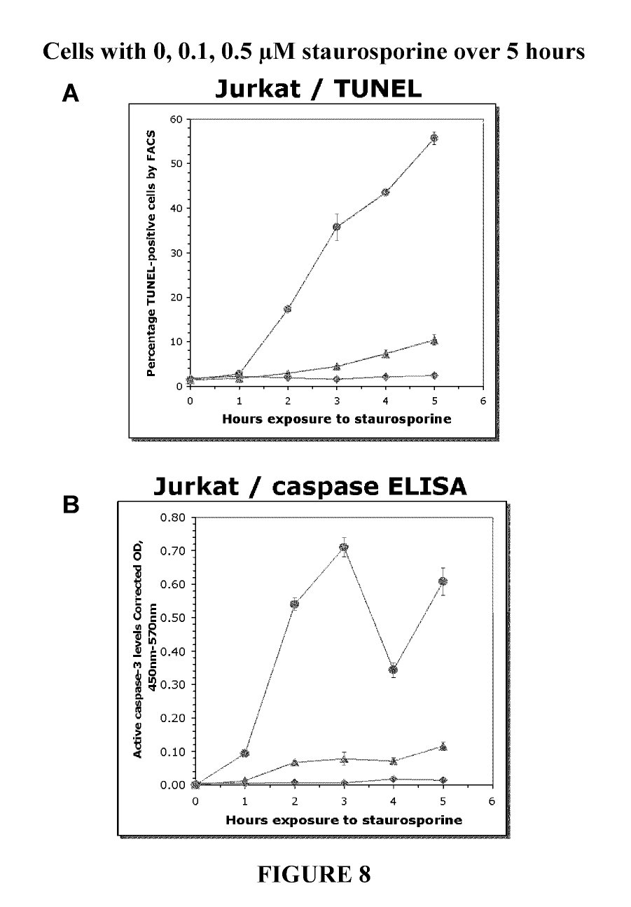

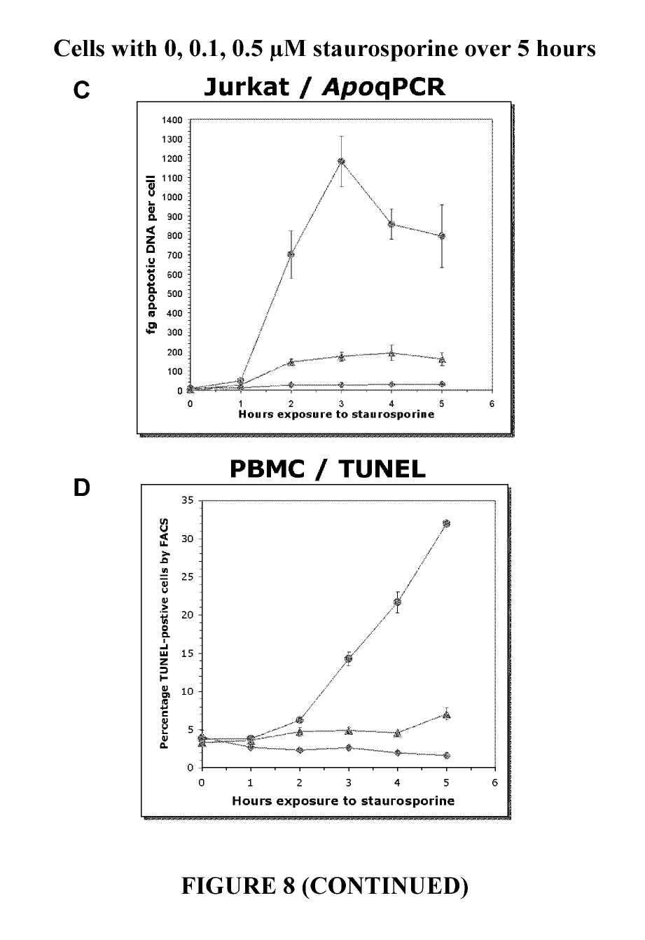

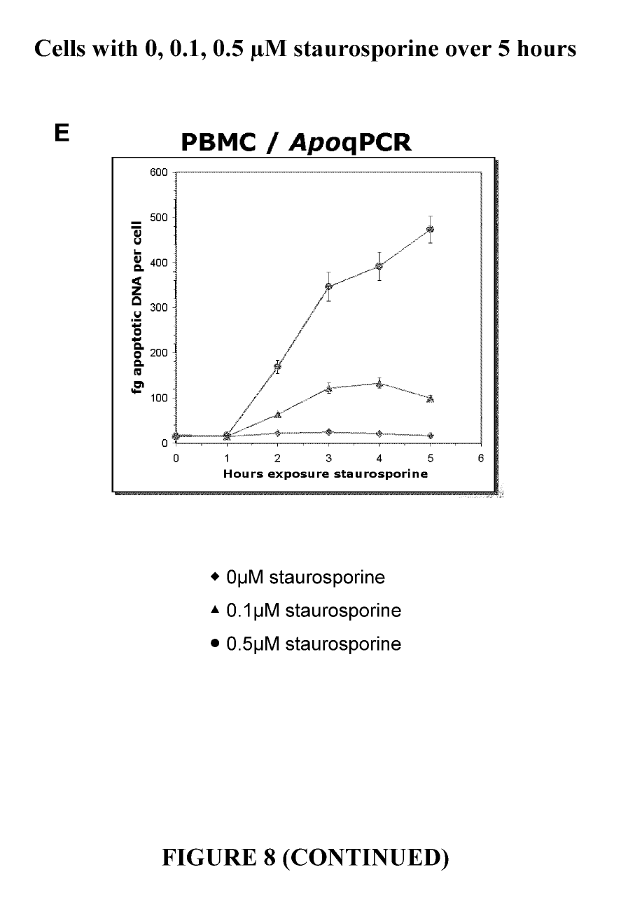

The products of qLM-PCR were electrophoresed to verify actual apoptotic fragment amplification over a broad range of target nucleic acid input amounts. Target nucleic acid ranged in input amount from 15000 pg to 14.64 pg per 25 μl reaction, in 2-fold dilutions. Reactions were terminated at 20 cycles (see FIG. 5) to detect products at or near the plateau of amplification, and then electrophoresed on 1.5% agarose gels. FIG. 6 shows consistent amplification of specific oligonucleosomal-sized apoptotic fragments.

### EXAMPLE 6

The Presence of High Molecular Nucleic Acid in qLM-PCR Reactions Does not Influence the Threshold Cycle Value

The generation of 100% apoptotic nucleic acid means that all standard curve Ct values are derived from reactions that do not contain high molecular weight genomic nucleic acid (typically 35-50 kbp after purification)—yet test samples, for example clinical sample genomic DNA, contains genomic nucleic acid of size 35-50 kbp because only a minor fraction of the purified nucleic acid is apoptotic. Experiments were conducted to determine if the presence of high molecular weight nucleic acid in qLM-PCR reactions influences Ct. One approach was to generate zero-apoptotic genomic nucleic acid by physical recovery of the 35-50 kbp fraction after electrophoresis, then spiking that nucleic acid into standard curve reactions. Unfortunately, even as a control (zero-apoptotic nucleic acid only), a significant Ct and apoptotic fragment amplification was achieved, indicating that complete physical separation of 35-50 kbp nucleic acid from lower molecular weight fragments was not possible (data not shown). A second approach was to mimic high molecular weight genomic nucleic acid with the presence of 48.5 kbp lambda phage genomic DNA. Annealing/ligation reactions with high, medium and low concentrations of target apoptotic DNA, and negative control (water) were spiked with lambda nucleic acid (New England Biolabs). Results show that Ct values achieved with this nucleic acid were not significantly different from those without, and that in fact a Ct generated with lambda nucleic acid alone was equivalent to that generated by a target nucleic acid amount approximately 1.3 logs lower than the least amount in the standard curve. The effect therefore lies beyond the lower limit of the standard curve (Table 2). Linear regression modeling of the raw data revealed that the apoptotic DNA amount is strongly associated with Ct as expected (p<0.001); and after correcting for apoptotic DNA amount, the presence or absence of X did not influence Cts (p=0.9).

For real time PCR for both apoptotic DNA quantitation (qLM-PCR) and CCRS housekeeping gene (Cell Number qPCR), runs were not accepted if the standard curve R² value was less than 0.985. For real time PCR runs for both apoptosis and CCRS housekeeping genes, mean Ct values of triplicate Cts were not accepted if the Ct range of triplicates spanned ≧1.0.

For both qLM-PCR and Cell Number qPCR arms, samples were run in triplicate and repeated to confirm values.

### EXAMPLE 7

### ApoqPCR: Annealing/Ligation (“Annealing/Ligation Reactions”) Construction of Apoptotic Standard DNA Dilutions

For production of apoptotic nucleic acid standards, the concentration of 100% Jurkat cell apoptotic nucleic acid in T[E/2] was determined spectrophotometrically (Nanodrop) and made to 23.15 ng/μl with T[E/2]. Six 4-fold dilutions in T[E/2] were then made starting from 9.26 ng/μl (40%). Single use aliquots of these standards were stored at −80° C.

### Annealing/Ligation:

For each standard, in a volume of 24 μl:

| | |
|---|---|
| 17.28 μl | nucleic acid (at 9.26 ng/μl to |
| | 9.043 pg/μl) |
| 0.96 μl | DHLMPCR1 24 mer (Sigma) at 0.005 |
| | nmol/μl in T[E/2]-AGCACTCTCGAGC |
| | CTCTCACCGCA; |
| | (SEQ ID NO: 1) |
| 0.96 μl | DHLMPCR2 12 mer (Sigma) at 0.005 |
| | nmol/μl in T[E/2]- |
| | TGCGGTGAGAGG |
| | (SEQ ID NO: 2) |
| 4.8 μl | 5X ligation buffer (with poly- |
| 24 μl total | ethylene glycol, Invitrogen) |

For each test sample, in a volume of 24 μl:

| | |
|---|---|
| 17.28 μl | Genomic nucleic acid (χ ng/μl, up |
| | to 400 ng total) |
| 0.96 μl | DHLMPCR1 24 mer (Sigma) at 0.005 |
| | nmol/μl in T[E/2] AGCACTCTCGAGCCT |
| | CTCACCGCA; |
| | (SEQ ID NO: 1) |
| 0.96 μl | DHLMPCR2 12 mer (Sigma) at 0.005 |
| | nmol/μl in T[E/2] LMPCR2-TGCGGTGA |
| | GAGG |
| | (SEQ ID NO: 2) |
| 4.8 μl | 5X ligation buffer (with poly- |
| 24 μl total | ethylene glycol, Invitrogen) |

For standards, test samples, and no template control (PCR grade water) 24 mers and 12 mers were annealed to form blunt-ended partially double stranded linkers by step-wise cooling from 55° C. to 15° C. in 5° C./8 minute increments then 10° C./20 minutes, employing a model PTC 200 DNAEngine (MJ Research, MA, USA). At the 10 minute point of 10° C. the program was paused and 2.4U T4 DNA ligase (Invitrogen, diluted to 1 U/μl in PCR grade water) was added, mixed, and the temperature continued for 10° C./10 minutes then ramped to 16° C./16 hours for ligation.

Subsequent to the ligation period, 24 μl annealing/ligation reactions were diluted to 80 μl with T[E/2], and 7.5 μl used in each of triplicate 25 μl qLM-PCR reactions.

### EXAMPLE 8

ApoqPCR: Apoptotic DNA Quantification Reactions (“qLM-PCR Reactions”)

25 μl qLM-PCR reactions were prepared containing 7.5 μl diluted annealing/ligation reaction, 0.625 μl additional 50 pmol/μl 24 mer (additional concentration of 1.25 pmol/μl final), 1× Taq polymerase buffer (67 mM Tris-HCl pH 8.8, 16.6 mM [NH₄]₂SO₄, 0.45% Triton X-100, 200 μg/ml gelatin; Fisher Biotech, Thebarton Australia), 320 μM (each) dATP, dTTP, dGTP, dCTP (Promega), 2 mM MgCl₂ (Fisher Biotech), 0.4× SYBR Green I (Invitrogen), PCR grade water and 0.1 U/μl Taq polymerase (Fisher Biotech) as a Taq-antibody complex allowing a PCR hot-start. The Taq-antibody complex was prepared with ‘Jumpstart’ Taq antibody (Sigma-Aldrich, Mo. USA) according to instructions, generating a complex at 0.83 (Taq) U/μl.

Cycling conditions were as follows: Employing a Stratagene MX3000P instrument (Agilent Technologies, Stratagene, La Jolla, Calif., USA) with software MXPro version 4.10, triplicate qLM-PCR reactions were heated to 94° C. for 1 minute to activate Taq polymerase and remove 12mers, then ramped to 72° C. for 4 minutes to re-anneal the target nucleic acid and generate complimentary sequence to the ligated 24 mers. PCR then proceeded over 40 cycles of 94° C. for 1 minute followed by annealing/extension at 72° C. for 3 minutes. Fluorescence data was collected at the end of annealing/extension steps. For each standard or test sample, one of 3 triplicate 25 μl reactions comprises:

| | | |
|---|---|---|
| 7.5 | μl | Diluted Annealing/Ligation |
| 0.625 | μl | DHLMPCR1 24mer (Sigma) at 50 pmol/μl in T[E/2] |
| 2.5 | μl | 10X Taq buffer (Fisher Biotech) |
| 3.2 | μl | 4X dNTP's (10 mM total) (Promega) |
| 2 | μl | 25 mM MgCl₂ (Fisher Biotech)) |
| 3 | μl | 2.5 U Taq/Antibody complex |
| 2.5 | μl | 4X SYBR Green I (Invitrogen) |
| 3.675 | μl | H₂O |
| 25 | μl total | |

### EXAMPLE 9

ApoqPCR: Cell Number Quantification Reactions (“Cell Number qPCR Reactions”)

Standards to estimate cell number equivalence of genomic nucleic acid were constructed. PBMCs from ˜100 ml blood were isolated by Ficoll density gradient centrifugation, washed 5× with room temperature PBS to remove platelets, resuspended in 1 ml PBS, accurately counted by haemocytometer (mean of 4 counts), and resuspended to 6×10⁷ cells/ ml in freshly prepared lysis buffer (10 mM Tris HCl pH 8.0, 1 mM EDTA, PCR grade water; final concentrations of 0.002% Triton X100, 0.002% SDS and 0.8 mg/ml proteinase K [Roche] added just before use). Cells in lysis buffer were incubated at 56° C. for 1 hour with occasional agitation, then proteinase K inactivated at 95° C. for 15 minutes. Standards were constructed by further diluting the cooled and mixed lysate from 50,000 cells worth/1.62 μl to 16 cells worth/1.62 μl, divided into single use aliquots and stored at −80° C. prior to use.

A 25 μl reaction volume was made comprising 1.62 μl (using a Gilson P2) of standard nucleic acid cell lysate, or the same sample genomic nucleic acid used for an apoptotic nucleic acid reaction, 200 nM (final) of LK46 primer, 200 nM (final) of LK47 primer 1× (final) of Brilliant II qPCR Master Mix (Stratagene) and PCR grade water. LK46 and LK47 primers span a 239 by region of the single copy human CCRS gene (Zhang et al., J Exp Med 190:725-732, 1999).

For each standard, in a volume of 25 μl:

| | | |
|---|---|---|
| 12.5 | μl | Brilliant II qPCR Master Mix (Stratagene) |
| 2 | μl | 2500 nM LK46 primer (Sigma) in T[E/2] |
| 2 | μl | 2500 nM LK47 primer (Sigma) in T[E/2] |
| 6.88 | μl | H₂O |
| 1.62 | μl | Standard nucleic acid cell lysate. |
| 25 | μl total | |

For each test sample, in a volume of 25 μl:

| | |
|---|---|
| 12.5 μl | Brilliant II qPCR Master Mix |
| | (Stratagene) |
| 2 μl | 2500 nM LK46 primer (Sigma) |
| | (GCTGTGTTTGCGTCTCTCCCAGGA |
| | (SEQ ID NO: 3)) |
| 2 μl | 2500 nM LK47 primer (Sigma) |
| | (CTCACAGCCCTGTGCCTCTTCTTC |
| | (SEQ ID NO: 4)) |
| 6.88 μl | H₂O |
| 1.62 μl | Genomic nucleic acid (the same |
| 25 μl total | test sample genomic nucleic acid |
| | used for apoptotic DNA |
| | quantitation in qLM-PCR reactions) |

Cycling conditions were as follows: Employing a Stratagene MX3000P instrument (Agilent Technologies, Stratagene, La Jolla, Calif., USA) with software MXPro version 4.10, triplicate Cell Number qPCR reactions for standards, test samples and no template control were heated to 95° C. for 10 minutes to activate DNA polymerase, followed by 40 cycles of 94° C. for 20 seconds, 58° C. for 30 seconds and 72° C. for 30 seconds. Fluorescence data were collected at the end of annealing steps. Dissociation curves established that a single product is synthesized for all standard dilutions.

Many modifications will be apparent to those skilled in the art without departing from the scope of the present invention.

TABLE 1
qLM-PCR apoptotic DNA standard curve:
Two-fold dilution series, 1000-fold dynamic range
Amount apoptotic DNA
(pg) in qLM-PCR reaction

15000.00
7500.00
3750.00
1875.00
937.50
468.75
234.38
117.19
58.59
29.30
14.65
No template control (NTC)

TABLE 2
High molecular weight DNA in qLM-PCR reactions does not influence the apoptotic DNA standard curve *

| | 3750 pg apoptotic DNA in | | 234 pg apoptotic DNA in | | 14.6 pg apoptotic DNA in | | No apoptotic DNA in | |
|---|---|---|---|---|---|---|---|---|
| | 25 μl qLM-PCR reaction | | 25 μl qLM-PCR reaction | | 25 μl qLM-PCR reaction | | 25 μl qLM-PCR reaction | |
| | With 1875 pg | Without | With 1875 pg | Without | With 1875 pg | Without | With 1875 pg | Without |
| | λ phage | λ phage | λ phage | λ phage | λ phage | λ phage | λ phage | λ phage |
| | gDNA | gDNA | gDNA | gDNA | gDNA | gDNA | gDNA | gDNA |
| Cts of | 14.07 | 13.47 | 18.36 | 18.74 | 22.97 | 22.72 | 26.46 | No Ct |
| individual | 13.94 | 13.87 | 18.48 | 18.36 | 22.79 | 22.87 | 26.33 | No Ct |
| reactions | 13.93 | 13.96 | 18.45 | 18.89 | 23.01 | 22.77 | 26.69 | No Ct |
| (n = 8) | 13.98 | 14.05 | 18.32 | 18.86 | 23.26 | 23.18 | No Ct | No Ct |
| | 14.21 | 14.01 | 18.65 | 18.89 | 23.15 | 23.67 | 26.15 | 36.86 |
| | 14.18 | 14.15 | 18.48 | 18.96 | 23.17 | 22.99 | 27.41 | No Ct |
| | 13.91 | 13.31 | 18.05 | 18.90 | 23.01 | 22.41 | 25.87 | No Ct |
| | 13.68 | 13.92 | 18.42 | 18.87 | 22.87 | 23.45 | 26.54 | No Ct |
| Mean Ct | 13.98 | 13.83 | 18.40 | 18.80 | 23.02 | 22.97 | 26.70 | No Ct |

* All Cts measured at fluorescence (dR) 2,500.

### BIBLIOGRAPHY

Altschul et al., Nucl. Acids Res. 25: 3389, 1997
Ausubel et al., eds., Current Protocols In Molecular Biology, John Wiley & Sons, Inc., Unit 19.3, 1995
Ausubel et al., eds., Current Protocols In Molecular Biology, John Wiley & Sons, Inc., pp. 2.10.1-2.10.16, 1995
Beaucage et al., Tetrahedron Letters 22: 1859-1862, 1981
Brown et al., Methods Enzymol. 68: 109, 1979
Chomczynski and Sacchi, Anal. Biochem. 162: 156, 1987
Christensen et al., Biochem J 354: 481-484, 2001
Doolittle, Annu. Rev. Biochem. 64: 287-314, 1995
Eggers et al., BioTechniques 17: 516-5240, 1994
Guo et al., Nucleic acids Res. 22: 5456-5465, 1994
Holstrom et al., Anal. Biochem. 209: 278-283, 1993
Hooker et al., J. Cell. Mol. Med. 13(5): 948-958, 2009
Hunter and Kearney, Biol Cybern 47(2): 141-146, 1983
Jeanmougin et al., Trends Biochem. Sci. 23: 403-5, 1998
Kawai et al., Anal. Biochem. 209: 63-69, 1993
Kerr and Harmen, Apoptosis: the molecular basis of cell death, Tomei and Cope ed, New York: Cold Spring Harbor Laboratory Press, pp.5-29, 1991
Liu et al., J. Am. Chem. Soc 118: 1587-1594, 1996
Maskos and Southern, Nucleic acids Res. 20: 1679-1684, 1992
Narang et al., Methods Enzymol. 68: 90, 1979
Newton et al., Nucleic acids Res. 21: 1155-1162, 1993
O'Connell-Maloney et al., TIBTECH 14: 401-407, 1996
Rasmussen et al., Anal. Biochem. 198: 138-142, 1991
Running et al., Biotechniques 8: 276-277, 1990
Sambrook and Russell, Molecular cloning, a laboratory manual. 3rd ed. Cold Spring Harbor, New York: Cold Spring Harbor Laboratory Press, 2001
Sambrook et al., eds., Molecular Cloning. A Laboratory Manual, Cold Spring Harbor Press, sections 1.101 to 1.104, 1989
Sooknanan et al. Biotechniques 17: 1077-1080, 1994
Staley et al. Cell Death and Differentiation 4: 66-75, 1997
Syvänen Anne-Christine, Nature Genetics, 2: 930-940, 2001
Tyagi et al., Proc. Natl. Acad. Sci USA 93: 5395-5400, 1996
Wallace et al., Nucl. Acids Res. 6: 3543, 1979
Wood et al., Proc. Natl. Acid. Sci. USA 82: 1585, 1985
Zhang et al., J Exp Med 190:725-732, 1999.

What is claimed is:

1. A method for quantifying apoptosis in absolute terms in a cellular sample by real-time PCR, the method comprising:
(a) obtaining first control genomic nucleic acid which is apoptotic nucleic acid from a cellular sample that is substantially 100% apoptotic;
(b) obtaining test genomic nucleic acid derived from a test cellular sample;
(c) subjecting a plurality of different known concentrations of first control genomic nucleic acid to real-time apoptosis-specific multi-product PCR in the presence of a nucleic acid-binding fluorophore or other detectable tag to obtain a threshold cycle number (Ct) or other equivalent value at different nucleic acid concentrations and establishing a linear numerical relationship between Ct or equivalent value and apoptotic nucleic acid concentration;
(d) subjecting test nucleic acid to real-time apoptosis-specific multi-product PCR in the presence of a nucleic acid-binding fluorophore or other detectable tag to obtain a Ct or other equivalent value; and
(e) establishing the quantity of apoptotic nucleic acid in the test nucleic acid, which corresponds numerically to the concentration of nucleic acid in (c) which determines the Ct or equivalent value obtained in (d).

2. The method of claim 1 wherein the detectable tag is a universal dye, such as SYBR Green I.

3. The method of claim 1 wherein the real-time apoptosis-specific multi-product PCR of steps (c) and (d) is ligation-mediated PCR (LM-PCR).

4. The method of claim 3 wherein LM-PCR comprises a linker comprising a nucleic acid sequence set forth in SEQ ID NO: 1 or a functional variant thereof

5. The method of claim 1 comprising:
(f) obtaining second control genomic nucleic acid derived from a cellular sample of known cell number;
(g) subjecting a plurality of different quantities of second control genomic nucleic acid corresponding to different cell numbers to real-time single copy gene specific PCR in the presence of a nucleic acid-binding fluorophore or other detectable tag to obtain a threshold cycle number (Ct) or other equivalent value at different nucleic acid concentrations and establishing therefrom a linear numerical relationship between Ct or equivalent value and cell number;
(h) subjecting test nucleic acid to the real-time single copy-gene specific PCR in presence of a nucleic acid-binding fluorophore or other detectable tag to obtain a Ct or other equivalent value; and
(i) establishing the cell number corresponding to the test nucleic acid which corresponds numerically to the number of cells in (g) determining the Ct or equivalent value obtained in (h).

6. The method of claim 5 further comprising determining the quantity of apoptotic nucleic acid and cell number as a function of Ct and expressing the quantity of apoptotic DNA as a function of cell number.

7. A standard composition comprising apoptotic nucleic acid as a predetermined proportion of an isolated nucleic acid sample.

8. The standard composition of claim 7 comprising substantially 100% apoptotic nucleic acid.

9. A kit comprising a standard composition comprising apoptotic nucleic acid as a predetermined proportion of an isolated nucleic acid sample, optionally sold together with instructions for use in a method for quantifying apoptosis in a cellular sample.

10. The kit according to claim 9 comprising a standard composition of isolated genomic nucleic acid comprising substantially 100% apoptotic nucleic acid.

11. The kit according to claim 9 comprising 100% apoptotic nucleic acid or another pre-determined percent apoptotic nucleic acid provided in a range of several different quantities in separate containers.


SEQUENCE LISTING (4 sequences)

SEQ ID NO: 1 - DNA, 24 bp, Artificial Sequence
  oligonucleotide primer
agcactctcgagcctctcaccgca

SEQ ID NO: 2 - DNA, 12 bp, Artificial Sequence
  oligonucleotide primer
tgcggtgagagg

SEQ ID NO: 3 - DNA, 24 bp, Artificial Sequence
  oligonucleotide primer
gctgtgtttgcgtctctcccagga

SEQ ID NO: 4 - DNA, 24 bp, Artificial Sequence
  oligonucleotide primer
ctcacagccctgtgcctcttcttc